(19) United States
(12) Patent Application Publication
(10) Pub. No.: US 2023/0346830 A1
(43) Pub. Date: Nov. 2, 2023

(54) TREATMENT OF A VIRAL PANDEMIC USING CIGARETTES WITH ADDED NITRATES

(71) Applicant: David H. Fine, Cocoa Beach, FL (US)

(72) Inventor: David H. Fine, Cocoa Beach, FL (US)

(73) Assignee: David H. Fine, Cocoa Beach, FL (US)

(21) Appl. No.: 17/797,608

(22) PCT Filed: Feb. 15, 2021
(86) PCT No.: PCT/US2021/018111

§ 371 Date: Aug. 4, 2022

Related U.S. Application Data
(60) Provisional application No. 63/041,772, filed on Jun. 19, 2020.
(60) Provisional application No. 62/977,594, filed on Feb. 17, 2020.

(51) Int. Cl. A61K 33/00; A24B 15/18; A24D 1/00; A24B 15/28; A61K 9/00

(52) U.S. Cl. CPC A61K 33/00; A24B 15/186; A24D 1/002; A24B 15/287; A61K 9/007

(57) ABSTRACT

A modified cigarette is used as a low-cost, widely available combustion engine to deliver high-dose nitric oxide (NO) directly into the lungs of people ill from a viral lung pandemic, similar to the influenza virus or the coronavirus.

### PRIORITY CLAIM

This application claims priority to U.S. Provisional Patent Application No. 63/041,772, filed Jun. 19, 2020 and U.S. Provisional Patent Application No. 62/977,594, filed Feb. 17, 2020, each of which is incorporated by reference in its entirety.

### TECHNICAL FIELD

The invention relates to low-cost modifications to cigarettes to deliver enhanced nitric oxide to treat patients during a coronavirus or influenza during a massive pandemic where millions of people are critically ill at the same time.

### BACKGROUND

Severe acute respiratory syndrome (SARS), was reported to be associated with a novel coronavirus (CoV), and was first identified during late 2002 in Guangdong Province, China. The mortality rate of SARS was reported to be from 6% to 55%. Coronaviruses are enveloped single-stranded positive-sense RNA viruses with genomes of about 27 to 30 kb. Coronaviruses belong to the family Coronaviridae, in which SARS CoV forms a distinct group within the genus Coronavirus.

### SUMMARY

In one aspect, a method of treating a viral pandemic in a patient can include administering a high dose of nitric oxide (NO) for a single breath to the patient, followed by a period of approximately 5 to 20 breaths of fresh air, equivalent to about one minute, where the NO concentration in the single breath is no less than about 1,500 ppm and, in certain circumstances, can be as high as 4000 ppm.

In another aspect, a method of treating a subject at risk of infection can include exposing the subject to an intermittent dose of NO gas, where a relatively massive dose of NO is used to break through the outer protective sheath of host cells where the virus is present, without the high NO concentration causing unacceptably high levels of methemoglobin, since the high NO concentration is for a single breath only, followed by multiple breaths of fresh air to clear some or all of the methemoglobin that may be formed.

In another aspect, a method of delivering a life saving drug directly into the lung of a sick patient can use a cigarette as a disposable drug delivery engine.

In another aspect, a treatment device can include a heat source and a nitric oxide source positioned to be heated by the heat source.

In certain circumstances, the NO can be delivered from a cigarette to which a chemical compound or additive has been added to boost the NO concentration in the inhaled smoke.

In certain circumstances, the NO can be formed when the cigarette is smoked and the NO is inhaled into the lungs.

In certain circumstances, the additive can be widely and cheaply available throughout the industrialized world as well as in third world countries.

In certain circumstances, the additive or compound can include an inorganic nitrate, for example, including nitrates of potassium, sodium, calcium and ammonia.

In certain circumstances, the additive can include a mixture of nitrate salts.

In certain circumstances, all the ingredients can be low cost and widely available, including the cigarettes and the additive or additives.

In certain circumstances, the nitrate salts can be commonly used nitrate fertilizers.

In certain circumstances, the nitrate salts can be used in food preparation and preservation.

In certain circumstances, the additives can be sprayed on the cigarette or the cigarette can be dipped in a concentrated solution of the additive.

In certain circumstances, the additive can be a fine powder which is applied to the outside of the cigarette.

In certain circumstances, the NO concentration that is delivered to the lung is sufficient for NO to enter through the protective wall of a host cell to where the virus is lurking inside the cell, thereby disrupting the replication of the virus inside the cell and slowing down or stopping the infection.

In certain circumstances, the drug can be manufactured during the combustion of the additive in the cigarette.

In certain circumstances, the drug can be nitric oxide which is manufactured in the cigarette by the decomposition of nitrates in the hot flame zone as the cigarette is smoked.

In certain circumstances, a regular cigarette can produce nitric oxide for inhalation at a high, intermittent concentration. The inhaled nitric oxide can lower the likelihood of a virus infection by inhalation. Intermittent bursts of high nitric oxide concentration in cigarette smoke of between 250 ppm and 1350 ppm in each puff can inhibit the replication cycle of a severe acute respiratory syndrome coronavirus.

In certain circumstances, the drug can be vaporized and inhaled by the hot smoke as the cigarette is smoked.

In certain circumstances, the treatment device can include a heat source that is a combustible product.

In certain circumstances, the combustible product can be a cigarette.

In certain circumstances, the nitric oxide source can include an inorganic nitrate.

In certain circumstances, the inorganic nitrate can include a salt coated on or imbedded in a cigarette.

The details of one or more embodiments of the invention are set forth in the accompanying description below. Other features, objects, and advantages of the invention will be apparent from the description and from the claims.

### BRIEF DESCRIPTION OF THE DRAWINGS

FIG. 1 depicts a schematic of a treatment device.

### DETAILED DESCRIPTION

Nitric oxide (NO) has been shown in vitro, to have an inhibitory effect on SARS CoV, together with other virus infections. NO is also known to be an important signaling molecule between cells. NO has also been shown to inhibit viral protein and RNA synthesis. The higher the level of NO, the more pronounced was the impact on replication. The promising in vitro findings did not translate to in vivo studies. Inhaled NO was delivered to influenza-infected mice either continuously at 80 ppm or intermittently at 160 ppm for 30 minutes followed by 3½ hours of breathing air. At these doses inhaled NO had no effect on the virus. No work has been carried out on treating viral lung infections with much higher inhaled NO doses such as 1000-2000 ppm because of the major concern that at these high NO concentrations the methemoglobin levels would be too high and quickly cause harm. This concern is unfounded and false, since all people who smoke cigarettes, cigars or pipes inhale NO at these very high concentrations, especially during the last puff, without being overcome by high methemoglobin, especially when only smoking occasionally. These high NO concentrations do not cause methemoglobin problems because even a chain-smoker breathes ambient air for approximately 60 seconds between puffs, which clears much of the methemoglobin from the body.

Thus, the intermittent dose that is known to be safely tolerated by adults is 160 ppm continuously for 30 minutes followed by 3½ hours of breathing clean air, or a massive dose of 2000 ppm for a single breath, followed by 5 to 20 breaths of fresh air. From a Gibbs free energy perspective and the perspective of the second law of Thermodynamics, the higher the NO concentration, the higher the driving force that is needed to penetrate the protective cell membrane where the virus is lurking. Once NO has penetrated the cell wall, then presumably the replication of the virus can be slowed or stopped, as shown in the in vitro work.

A modified cigarette is used as a low-cost, widely available combustion engine to deliver high-dose nitric oxide (NO) directly into the lungs of people ill from a viral lung pandemic, similar to the influenza virus or the coronavirus. In one example, a cigarette is modified by spraying with or dipping in a solution containing nitrate salts of the type used in widely available fertilizers and some widely used food additives. The cigarette, when smoked and inhaled, then delivers a high dose of NO at a concentration of about 2000-4000 parts per million (ppm), which is sufficient to penetrate the host cells which the virus has infected, thereby slowing or stopping the replication of the virus.

A cigarette can be readily modified to deliver high sustainable NO concentration in the 2000-4000 ppm range, while providing the ideal timing sequence for the delivery of intermittent high inhaled NO concentrations. The advantage of using a conventional cigarette for the drug delivery is that they are widely available worldwide, including in third world countries, and are also relatively inexpensive.

Because of the numerous downside risks from smoking, the treatment option described here for severe viral infections is intended for use as a last resort in the event of a catastrophic country-wide or world-wide pandemic emergency, where tens of millions of people are desperately ill at the same time, and conventional medical services and options are completely overwhelmed. It is not intended that it be used under normal conditions when conventional medical services are available and functioning properly.

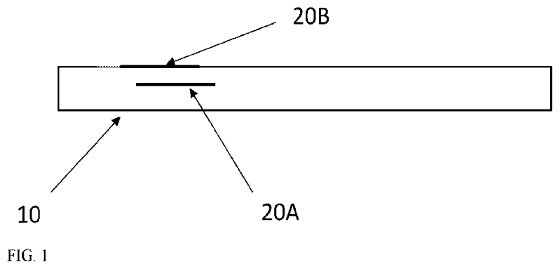

Referring to FIG. 1, a heat source 10 can include a nitric oxide source 20A or 20B on a surface of, or, optionally, internal to, the heat source 10. The heat source 10 can be, for example, a cigarette, a cigar or other combustible device. The nitric oxide source can be a nitrate salt, for example, an inorganic nitrate salt. The nitric oxide source can be sprinkled, sprayed or soaked into the heat source. Other methods of depositing can be used.

Severe acute respiratory syndrome (SARS), was reported to be associated with a novel coronavirus (CoV), and was first identified during late 2002 in Guangdong Province, China. The mortality rate of SARS was reported to be from 6% to 55%. Coronaviruses are enveloped single-stranded positive-sense RNA viruses with genomes of about 27 to 30 kb. Coronaviruses belong to the family Coronaviridae, in which SARS CoV forms a distinct group within the genus Coronavirus. Coronaviruses are named for their crown-like spikes when viewed through a microscope. In 2019/2020 there was an outbreak starting in China of a new strain called 2019-nCov.

The 2019 nCoV outbreak is an example of how a new strain of the influenza type virus can cause worldwide panic as the number of people infected grows rapidly. The 2019-nCOV situation has all of the ingredients of how a pandemic can occur. A new virus appears that is transmitted from an animal host to man. The virus then spreads rapidly from person to person. The virus was reported to be highly infectious before symptoms are developed, so no one knew whether they were infectious or not, or had been in contact with someone who had been infected. This situation can lead to an explosive spread of the new disease.

The mathematics of the explosive growth in a pandemic are almost identical to that of three other types of common explosive growth, a chemical explosion, a nuclear explosion and the sudden explosive growth of a pestilence. In a chemical explosion, a reaction gives of heat, which makes the reaction go faster and it gives off more heat at a faster and faster pace until an explosion occurs. The latency period can be micro to milliseconds for an explosive like TNT to months for bags of wool or oily rags. Fine (1967) observed oscillatory conditions before a gas explosion under laboratory conditions in which the explosion started to proceed and then slowed, with the cycle repeating until it died out or resulted in an explosion. A nuclear chain reaction can be controlled as in a nuclear power reactor or be designed to be an atomic bomb which occurs when a critical mass of radioactive material is present. In a pestilence explosion, such as occurred with the gypsy moths in the US northeast in 1981, the latency period can be measured in years to decades, with several significant episodes before one year the gypsy moth population suddenly explodes and devastates hardwood trees and large forests. In a viral pandemic, the latency period is likewise expected to be slow over several months before it suddenly reaches a critical mass of people infected and exposed. If the virus has a high fatality rate, then the ensuing pandemic can kill tens to hundreds of millions of people worldwide. With so many people desperately sick at the same time, in the same place, it becomes impossible to treat the sick using conventional medical resources. Even if in-hospital treatment options are developed and are available, there are just not enough medical personnel to attend to and treat the vast number of desperately sick and highly infectious patients.

It is essential therefore to have a simple, low cost and effective way to treat tens of millions of people at the same time, without access to medical personnel or hospitals. In addition, the drug needs to be able to treat new strains of the virus, unlike highly strain-specific vaccines.

Vaccines to the new virus strain will eventually be developed, but the problem remains of how to treat the tens of millions of people who are infected at the same time, before a viable vaccine can be developed.

Ideally, the need is for a low-cost treatment procedure that is safe and simple to use, and that is widely available in all locations and in all countries, including populations in the so called third world. The cost must be low, preferable less than a few US dollars per patient. The treatment should be effective at not only treating those that are already infected and with clear symptoms, but also effective prophylactically at treating those that are not yet symptomatic.

Inhaled nitric oxide (NO) has the potential of being such a candidate drug, provided that it can be shown to be effective in vivo and, most importantly, that it can be made widely available at a trivial cost. NO already has been shown by Åkerström et al (2005) to stop the in vitro replication of SARS strain of the Coronavirus. A critical advantage of NO is that it is delivered by inhalation directly to the lungs, the very site of the infection.

There are currently two pharmaceutical companies in the US that have FDA approval for delivering inhaled NO. For both companies, the FDA approval is for the treatment of Persistent Pulmonary Hypertension of the Newborn (PPHN). It is estimated that as much as 80% is for “off-label” use for a multitude of diseases. Because of complexity and safety, at the present time it is used exclusively in a Hospital and in an Intensive care facility.

Pharmaceutical grade NO gas sources require extensive electronic controls for delivery and safety.

As described herein, a novel but effective and very inexpensive way to deliver very high concentrations of NO to a patient's lungs for widespread mass use for treating patients in a pandemic, when minimal to zero trained medical supervision is available. The method modifies conventional cigarettes and/or cigars or any combustible tobacco products to deliver the high concentration of NO directly to the lungs. This is achieved by spraying, or soaking or impregnating a conventional cigarette, cigar or other tobacco product with a widely available nitrate fertilizer or food preservative, to boost the NO concentration when the product is smoked and inhaled. The technology is based on detailed knowledge and understanding of how NO is formed during combustion of tobacco at the flame front. While tobacco is the preferred combustible, other materials such as other plant leaves or even cellulose can be used if tobacco products were not available or desirable, for any reason.

NO Formation During Burning

NO is formed during combustion of a fuel where the fuel burns at a high temperature. There are two very distinct chemical mechanisms of how NO is formed in a flame.

First, if the temperature in the flame front reaches about 1800° C. or higher, then there is enough energy to begin to dissociate the N₂ in the air into N-atoms. It takes 226 kcal/mole to break the N₂ triple bond, which is one of the strongest of chemical bonds. The energy required is almost double that required to break O₂ into O-atoms. The N-atoms react with the O-atoms formed from the dissociation of O₂ gas and NO is formed.

In all flames, since the temperature is very high, NO is the thermodynamically stable oxide of nitrogen. As the gas cools, NO₂ is formed by the oxidation of NO with ambient O₂ from the air. At room temperature, NO₂ is the stable oxide of nitrogen. The homogeneous gas phase reaction of NO with O₂ that produces NO₂ was first studied by Bodenstein and Wachenheim in 1918. They showed the rate of formation of NO₂ is first order in O₂, second order in NO and third order in total pressure.

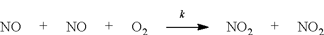

This equation has been widely used to calculate the rate of NO₂ formation from the known NO and O₂ concentrations.

Second, NO formation occurs when the fuel itself contains some N-atoms. This is typically in the form of an organic-N or inorganic-N impurity, which contains N-atoms in the molecule. The formation of NO from fuel-N can occur at the high flame temperatures of over 1800° C. Fine et al (1971) have shown that most of the high NO emissions from coal fired power stations was due to the N-content in the coal. The higher the N-content of the coal, then the higher the emissions of NO from the plant. Similarly, organic-N compounds in various fuels have been shown by Fine et al (1974) to be the primary source of the NO in the gas emissions from these combustion sources.

However, some flames burn at temperatures below 1800° C. Under these conditions, the flame is not hot enough to dissociate N₂ into N atoms so NO cannot be formed from N₂ atoms in the air. Forcing the peak flame temperature to be below about 1800° C. is one of the key engineering design requirements that are essential in so called low NOₓ combustion engines and furnaces.

Tobacco products, such as cigarettes, smolder at about 400° C. and burn with a flame temperature of about 800 to 900° C. During puffing, the temperature in the flame front can rise to about 1000° C. as air is sucked in and air is forced through the flame front. This is still nowhere near hot enough for N-fixation from air, as it takes about 1800° C. to break the N—N triple bond to form the required free N-atoms. The only source of N in NO from tobacco products is therefore from the N-containing compounds in the tobacco, where the N—C or N—H bonds are far weaker. There are two sources of N-compounds in tobacco, the organic-N compounds such as nicotine and the inorganic nitrates, primarily from the fertilizers that are used to grow the tobacco. Widely used fertilizers for tobacco are potassium, sodium and calcium nitrate. These nitrate based fertilizers are sold under various trade names worldwide.

Smoke from cigarettes has been shown to contain NO at peak concentrations of between about 100 ppm and 2000 ppm. The wide variation depends upon both the brand (UK Department of Health, 1998), the puff number (Fine, unpublished work) and the amount of nitrate in the tobacco.

Borland et al (1985) found that fresh cigarette smoke contained up to 1000 ppm of NO per puff. They stated that the yield is largely dependent on the nitrate content of the tobacco used in manufacture and hence is higher for U.S. blended and dark air-cured varieties than for cigarettes manufactured from Bright (Virginia) or Oriental tobacco.

The first puff contains the lowest instantaneous NO concentration, and the last puff the highest instantaneous concentration. The only oxide of nitrogen in the mainstream smoke (which is the smoke that is inhaled) is NO, and the NO₂ concentration that is inhaled has been shown experimentally to be very low or undetectable. Considerable NO₂ is formed in the side stream smoke in the room, as the smoke slowly ages with time.

The increase of NO in the puff sequence is presumed to arise from relatively stable organic N-compounds such as nicotine, which tend to distill ahead of the flame front. Thus, as the cigarette is smoked, the concentration of these compounds increases in the unburned tobacco, and more and more NO is formed as it is overtaken by the flame front. The nitrate containing salts of potassium, sodium and calcium nitrate, that are widely used in growing tobacco crops, decompose in the hot flame to produce NO, as shown in the Table:

| | | | |
|---|---|---|---|
| KNO₃ | Mwt 101 | Melting point 334° C. | Boiling point: 400° C. |
| | | | (decomposes) |
| NaNO₃ | Mwt 85 | Melting point 308° C. | Boiling point: 380° C. |
| | | | (decomposes) |
| Ca(NO₃)₂ | Mwt 164 | Melting point 561° C. | Decomposes when |
| | | | incandescent |

The use of cigarettes as a vehicle to deliver a life saving drug is not obvious and is counter intuitive. Cigarette smoke contains over 7,000 chemical compounds, including arsenic, formaldehyde, hydrogen cyanide, lead, nicotine, carbon monoxide, acrolein, and other poisonous substances. Over 70 of these are carcinogenic. Additionally, cigarettes are a frequent source of deadly fires in private homes, which prompted both the European Union and the United States to require cigarettes to be fire-standard compliant. Indeed, the approach of delivering a life saving drug by means of a cigarette is one of the last things that a medical professional would contemplate, and this patent will certainly be viewed as medical madness by the vast majority of health care professionals, and for good reason. Smoking leads to disease and disability and harms nearly every organ system of the body. It is the leading cause of preventable death according to the Center for Disease Control (CDC). Smoking can cause fatal diseases such as pneumonia, emphysema and lung cancer. Smoking causes 84% of deaths from lung cancer and 83% of deaths from chronic obstructive pulmonary disease (COPD). Smoking cigarettes can have many adverse effects on the body. Some of these can lead to life-threatening complications. In fact, according to the CDC, smoking cigarettes increases the risk of dying from all causes, not just those linked to tobacco use. Smoking cigarettes affects the respiratory system, the circulatory system, the reproductive system, the skin, and the eyes, and it increases the risk of many different cancers.

However, despite all these many well established health concerns and taboos, the reasons for selecting tobacco products such as cigarettes and cigars and pipe tobacco as the delivery vehicle, is because there is no other way, and also because the harm from smoking just a few cigarettes is minimal.

Consider:
Cigarettes are widely available throughout the US and the rest of the world, including in third world countries.
The cost per cigarette can be low. It varies from about 10 cents per cigarette to as much as 50 cents per cigarette, depending upon local taxes.
Cigarettes can provide a relatively well controlled combustion engine for the delivery of inhaled NO, and the delivery is directly into the lungs, right where it is needed.
It is anticipated that a treatment regimen may involve smoking a few cigarettes per day for a few days to a week. If faced with a choice of the very sick patient dying from the virus pandemic, it would be criminal medical malpractice to deny giving a life saving drug to tens of millions of people.
The medicinal use should not be habit forming since it is short lived. All of the negatives of smoking cigarettes are for prolonged daily use of cigarettes over many years.
The direct health risk from smoking just a few cigarettes is very low to nil. No one has suggested that there is a direct health risk from smoking just a few cigarettes.
It is the only practical way to deliver inhaled NO in a well controlled dose to millions of people with minimal to zero medical supervision.
It has the potential to save millions of lives in a full blown viral pandemic when conventional medical treatment options have been overwhelmed or have become non-existent.

Because of the numerous downside risks from smoking, the treatment option described in this patent is intended for use only in the event of a catastrophic pandemic, when tens of millions of people are infected and are desperately ill at the same time and medical services are completely overwhelmed. It is not recommended that it be used under normal conditions when conventional medical services are readily available. However, there may be situations under normal conditions when medical personnel may choose to deliver NO, or other possible drugs, by means of cigarette smoke, but it is unlikely.

A cigarette, as well as a cigar, are carefully engineered disposable combustion engines. They are designed to burn tobacco in a controlled manner and to deliver essentially the same ingredients in the smoke to the lungs of the smoker (Senneca et al 2008). They are manufactured in factories by automated production machinery that makes each cigarette virtually identical. The only minor difference from cigarette to cigarette is the slight variation in the chopped up tobacco leaves. Once the cigarette has been lit, the hot tip is designed to smolder at a temperature of about 400° C. The hot combustion products from the smoldering zone are discharged into the ambient air as a vertical column of warm air. When the smoker sucks on the cigarette, the smoke is drawn into the mouth and inhaled, a procedure commonly called puffing. As the cigarette is puffed, the hot smoldering gases, now enriched with oxygen from the air, are drawn through the unburned tobacco at the flame tip of the cigarette. At the edges, near the paper, there is an excess of oxygen. However, in the center where the bulk of the “fuel” resides, the oxygen is quickly consumed and the oxygen level is depleted. The gases that are inhaled therefore, contain not only the complete products of combustion of the tobacco, but also the fuel rich combustion products such as carbon monoxide, and the partially burned products and the products together with pyrolyzed products where there is very little to no oxygen. In addition, there are a multitude of vapors from the multitude of compounds that are vaporized by the hot gases as they pass through the tobacco column, especially, those that are close to the flame front, where the temperature is highest. Compounds which have relatively low boiling points are vaporized in front of the flame front. Some of these condense on the unburned tobacco, to be re-vaporized as the flame front approaches. For this reason, the concentration of many compounds in the smoke increases with each puff. During puffing, the flame temperature has been measured at between 800° C. and 1000° C., with most workers reporting an average flame temperature of about 900° C.

Various studies, including those of Norman et al. (1983) have shown that the NO concentration in the smoke is not dependent upon the total N-content of the tobacco, but rather only on the nitrate content. It has also been shown that between 75 to 90% of the NO from a cigarette is present in the side stream smoke and is not inhaled by the smoker in the mainstream smoke. This is presumably due to the fact that most organic N-containing compounds will tend to vaporize ahead of the flame front, and are inhaled together with their decomposition products. Nitrate salts of potassium, sodium and calcium, on the other hand, which are the nitrate salts that are widely used in the fertilizer that is used to grow the tobacco, decompose at the temperatures in both the smoldering and combustion zones, to form NO. Another reason for the mainstream smoke containing such a small fraction of the total NO generated by the cigarette is because puffing represents only 2 to 3 seconds per 60 seconds (3-5%) of the smoke that is generated.

Cigarette Modification

In order to deliver the required dose of NO, it will be necessary to add N-containing compounds to the cigarette. Nicotine and other complex N-containing compounds were considered and then abandoned as candidate N-compounds. The reasons why N-containing organic compounds were not selected as the optimum candidates are several:
Nicotine and other complex N-organics are relatively expensive and not widely or readily available in pure form to most people.
The decomposition and reaction products of nicotine and other complex organic compounds may be harmful when inhaled.
The nicotine content in a cigarette varies greatly from one brand to the next. On the low end, a single cigarette may contain about 6 milligrams (mg) of nicotine. On the high end, about 28 mg. The average cigarette contains about 10 to 12 mg of nicotine, with the average being 11 mg. If all the 11 mg of nicotine burned and all of the N was converted to NO, it would produce 1.9 mg of N gas or 4.08 mg NO, in the gas phase. The data from the UK study showed that the fraction of the N in the nicotine that is converted to NO is between 0.5 to 7%. This compares to 26 to 56% conversion efficiency from inorganic nitrate salts.
The conversion efficiency is reduced even further because of the fact that only 10 to 25% of the total NO that is produced is present in the mainstream smoke, with the balance being in the side stream smoke, which is not inhaled.
Norman et al also showed that the conversion of NO as inorganic nitrates to NO gas in the Main Stream (MS) and Side Stream (SS) smoke is efficient and varies from about 26% to 65%. This conclusion comes from the interpretation of the data in Table 6 from the UK study and in Table 1 from Norman et al (1983). See also Borland and Higenbottam, (1987). A typically cigarette weighs approximately 1 gram of which the tobacco content can vary between 65-100% depending on the type of cigarette, but it is usually closer to 90%. A cigar weighs approximately 2 grams and contains a similar proportion of tobacco as does a cigarette. The average cigarette has about 0.85 g to 0.9 g of tobacco.

There are several approaches for modifying cigarettes to increase the NO content of the main stream smoke. For the sick patient in the midst of a major pandemic, the procedure has to be easy and simple, similar to cooking food or baking bread, biscuits or a cake. Furthermore, the chemicals and the tools need to be readily available. More complex processes can be used if the nitrate addition is made in a factory setting, either during production of the cigarette or after sale by reworking the cigarettes in a factory setting.

Calcium nitrate is a white powder which is hygroscopic and highly soluble in water, 1212 g/L. It is widely used as a nitrate based fertilizer, especially in greenhouses and for hydroponics. It is sometimes formulated with ammonium nitrate as the double salt, called calcium ammonium nitrate. Liquid formulations are also available. An anhydrous, air-stable derivative is the urea complex Ca(NO₃)₂·4[OC(NH₂)₂], which has been sold as Cal-Urea. Potassium nitrate is a white to gray powder that occurs in nature as a mineral, niter. Unlike calcium and sodium nitrate it is not hygroscopic and is only partially soluble in water. It is a source of nitrogen, from which it derives its name. Potassium nitrate is one of several nitrogen-containing compounds collectively referred to as saltpeter. Major uses of potassium nitrate are in fertilizers, the manufacture of gunpowder and in processed meats where it combines with hemoglobin to give processed meats their red color. Sodium nitrate is hygroscopic and highly soluble in water. It is a white solid. It is a widely used as a fertilizer, and also used in pyrotechnics, as a food preservative and in glass and pottery enamels.

For use by the general population, depending on the nitrate salt that is available, a concentrated aqueous solution can be sprayed on the cigarette or the cigarette can be dipped into the concentrated solution. It should be dried before smoking. Another possible procedure for adding the nitrate salt could be to moisten the outside of the cigarette and then roll the cigarette in the dry powder, much like is done in food preparation. Another approach would be to roll their own cigarette using the tobacco from the cigarette or purchased tobacco. For home made cigarettes another technique would be to sprinkle the nitrate salt directly on the tobacco or on the cigarette paper.

The ideal amount of nitrate needed is about 1 to 4% by weight. If the dry powdered nitrate salt is to be added directly to the cigarette this can be achieved by adding approximately 1 gram of dry nitrate salt to a pack of 20 cigarettes, to achieve 50 mg per cigarette. It is usually easier and more reproducible to add the nitrate salt as a solution. At 20° C. the solubility of calcium nitrate is 12 g in 10 ml of water, for sodium nitrate the solubility is 10 g in 10 ml of water and for potassium nitrate the solubility is 2.4 g in 10 ml of water. Adding 0.1 ml of the saturated solution at 20° C. across the length of the cigarette, excluding the filter, will provide the needed amount of nitrate. For better accuracy the saturated solution described above can be diluted 10:1 to provide 100 ml of dilute solution, and then add 1 cc of liquid to each cigarette. A simple way of adding the solution would be to use a syringe or an eye dropper, for example.

Effect of Nitric Oxide on the Coronavirus (CoV) that was responsible for Severe Acute Respiratory Syndrome (SARS)

Severe acute respiratory syndrome (SARS), was reported to be associated with a novel coronavirus (CoV), and was first identified during late 2002 in Guangdong Province, China. The mortality rate of SARS was reported to be from 6% to 55%. Coronaviruses are enveloped single-stranded positive-sense RNA viruses with genomes of about 27 to 30 kb. Coronaviruses belong to the family Coronaviridae, in which SARS CoV forms a distinct group within the genus Coronavirus. Coronaviruses are named for their crown-like spikes when viewed through a microscope. In 2019/2020 there was a new outbreak in China of a strain called 2019-nCoV.

Nitric oxide (NO) has been shown to have an inhibitory effect on SARS CoV, together with other virus infections. NO is also known to be an important signaling molecule between cells. Åkerström et al (2005) reported that an organic NO donor, S-nitroso-N-acetylpenicillamine (SNAP), significantly inhibited the replication cycle of SARS CoV in a concentration-dependent manner. The higher the level of NO, the more pronounced was the impact on replication. They also showed that NO inhibits viral protein and RNA synthesis. SNAP inhibited the replication cycle of SARS CoV in a dose-dependent manner. Treatment with 100 μM SNAP resulted in a 2-log reduction in the yield of progeny virus, and the inhibitory effect was μeven more pronounced with 400 μM SNAP. The inhibitory effect of NO on SARS CoV infection in Vero E6 cells was further demonstrated by an immunofluorescence assay and Western blotting. They demonstrated that viral RNA production was significantly inhibited by 400 μM SNAP. The measurement of NO levels demonstrated that the concentration of nitrite produced by the cytokine treatment reached approximately the same level as that seen with 50 μM SNAP. They also observed the same level of inhibition of the virus replication cycle with 50 μM SNAP as that with the cytokine treatment. Their results demonstrated that NO specifically inhibits the replication cycle of SARS CoV, most probably during the early steps of infection, suggesting that the production of NO by iNOS results in an antiviral effect. They also noted that the production of NO should be adjusted to exert antiviral rather than damaging effects.

Darwish et al (2012) later showed that the in vitro findings of Åkerström et al (2005) and others did not translate to inhaled NO delivered to influenza-infected mice either continuously or intermittently at 80 or 160 ppm, respectively, using both prophylactic and post-infection treatment strategies. Inhaled NO was administered starting 1 hour prior to influenza WSN/33 infection and continued either continuously 24 hours per day at 80 ppm or intermittently at 160 ppm for 30 min every 3.5 hours. The inhaled NO was administered both prior to and for 5 days post-infection. Essentially, their in vivo treatment on mice infected with an influenza virus failed.

They did their study based on the fact that in vitro NO antimicrobial activity has been demonstrated against a variety of viruses including ectromilia virus, vaccinia virus, herpes simplex type 1 viruses, coronavirus, and influenza A and B viruses. In these studies, administration of the NO donor SNAP to virus-infected cells significantly reduced viral burden. A human trial for treatment of severe acute respiratory syndrome SARS found inhaled NO, at 30 ppm or less, decreased the spread and intensity of lung infiltrates and improved arterial oxygen saturation. Severe cases of influenza infection are often associated with multisystem organ failure and hypoxemic respiratory failure, including acute lung injury/acute respiratory distress syndrome requiring advanced mechanical ventilatory support. Affected individuals may receive ‘rescue’ therapies, including inhaled NO, in an attempt to improve outcome, although inhaled NO administration for ARDS secondary to viral pneumonia has not been specifically reported to improve clinical outcome. Darwish et al reported that inhaled NO administered prophylactically or post-influenza infection failed to improve survival of infected mice. No difference in lung viral load was observed between experimental groups. Although NO has antiviral activity against influenza A virus in vitro, in their study inhaled NO therapy, at the concentration and length of time delivered, provided no apparent benefit when used for treatment of influenza A virus infection in vivo in mice.

Inhaled NO is approved for treating term and near-term neonates with hypoxemic respiratory failure up to a dose of 80 parts per million (ppm). Darwish and others have shown that gaseous NO at a dose of no less than 160 ppm and with five hours of continuous exposure, could elicit a non-specific antimicrobial response against a broad range of microorganisms in vitro. They assumed that in vivo, 160 ppm of inhaled NO treatment, delivered for 30 minutes followed by a break of 3½ hours to breath air only, was the highest dose that a mammal could inhale in order to prevent NO binding to hemoglobin to form methemoglobin, resulting in reduced oxygen transport and hypoxemia, as well as the potential for elevated levels of the harmful NO metabolite NO₂. The same researchers, Miller et al. had shown that inhaled NO in an intermittent delivery regimen of 160 ppm for 30 min every 3.5 hours could prevent methemoglobinemia and reduce the potential of host cell toxicity in vitro and in vivo, while retaining antimicrobial properties in vitro.

The issue that Miller et al and other workers did not appreciate was that very much higher concentrations than 160 ppm can be safely given to mammals provided that they are allowed to breathe air for a multiple breaths between each dose. This information comes from Borland et al (1985), Norman and Keith (1965) and UK Department of health 1998, who have all shown that the NO concentration in a conventional cigarette can average 1000 ppm and be as high as 2000 ppm in the last puff (Fine, unpublished work) of a conventional cigarette. The reason that a smoker can tolerate a NO dose of >1000 ppm is presumably because the relatively massive NO concentration is present for just one breath, followed by 8 to 12 breaths of fresh air. That is how cigarettes are smoked and the smoker would be asphyxiated and pass out if every single breath was a full inhale of a puff.

What Darwish et al did not know was that it was incorrect to assume that a person could only tolerate a maximum dose of 160 ppm for 30 continuous minutes, which they called intermittent, followed by a break of 3½ hours to breathe free air. The break for breathing fresh air was for the body to recover from the methemoglobin that had been formed. The assumption was only valid if the inhaled NO was given continuously by means of a ventilator or nasal cannula, which is the procedure normally used for the delivery of inhaled gases. However, very much higher doses than even 160 ppm can be given if the mammal is allowed to take multiple breaths of fresh air in between every dose. Instead of defining an intermittent dose as a maximum of 160 ppm for 30 minutes as described by others, unexpectedly and advantageously, an “intermittent dose” described herein for the methods of the invention require greater than 1600-2000 ppm in a single breath followed by multiple breaths of breathing free air. The dose for a single breath can even be considerably higher than 1600 ppm of NO for a single breath, provided that it is followed by a period of breathing fresh air. Indeed, Fine has shown that some cigarettes deliver as high as 2000 ppm or more of NO in the last puff. For medicinal purposes the NO concentration in a puff could be considerably greater than even 2000 ppm, even as high as 3,000-4000 ppm or higher, provided that the person can clear the methemoglobin that will be formed by having multiple breaths of fresh air, multiple refers to at least 5 to 6 breaths and possibly as many as 20 or more. The typically time between puffs should be at least one minute and the number of breaths depends on the person and their ability to breathe freely. The NO₂ concentration in the mainstream smoke is very low to zero.

The intermittent dose of the inventions that can be safely tolerated seems to be at least 2000-3000 ppm or higher for a single breath, followed by 5 to 20 breaths before the next dose (puff). From a Gibbs free energy perspective and the second law of thermodynamics, the higher the NO concentration the higher the driving force that is needed to penetrate the protective cell membrane where the virus is lurking. Once NO has penetrated the cell wall, then presumably the replication of the virus can be slowed.

Dosing

The sustained dose for the treatment of infectious lung diseases is about 160 ppm of NO. This is a relatively high dose compared to the use in PPHN, where the typical dose is 20 ppm. With high sustained dosing of inhaled NO, methemoglobin is formed. Conventional medical practice includes monitoring of the methemoglobin so as to ensure that it does not get too high. For the use with cigarettes as the delivery engine, sustained dosing will not be used. Instead, the dosing will be transient, which is totally different. The patient will take a puff and receive an inhaled bolus of NO whose concentration will be of the order of 1000 to 2000 to 4000 ppm or even higher, together with other conventional compounds in smoke at the same concentrations as for conventional cigarettes. Only the NO concentration will be elevated. The patient would then breathe ambient air for a minute or more before taking the next puff. Breathing air in between puffs is essential so as to allow the methemoglobin to clear. The second puff will again deliver approximately the same high NO dose and again it is essential that the patient breathe for about a minute or more before taking the next puff. This is repeated 5 to 8 times, or until the cigarette has been used up. It is not intended that the sick patient chain smoke one cigarette after the other. Instead, there may be many minutes to an hour or more before the next dosing regimen, giving plenty of time for the methemoglobin to return to base levels. It will be important to keep the smoking rate down so as to minimize the build up of methemoglobin, especially when the methemoglobin concentration is not being measured.

The higher transitory concentration of 1000 to 2000 ppm to 3000 to 4000 ppm can be helpful in reaching and interfering with the virus.

References, each of which is incorporated by reference in its entirety.

### US Patent Documents

| | | |
|---|---|---|
| 5,418,170 | May 1995 | Rounbehler |

### OTHER REFERENCES

Fine, D. H., Williams, G. C., and Sarofim, A. F., “Nitrogen in Coal as a Source of Nitrogen Oxides,” Paper presented at the 71st National Meeting of the AIChE, San Francisco, December 1971.
Fine, D. H., Slater, S., Williams, G. C., and Sarofim, A. F., “Organic Nitrogen as a Source of Nitric Oxide in Flames,” Fuel, 53:120 (1974).
Fine, D. H. Spontaneous Ignition and Thermal Explosions; Ph.D thesis. Leeds University, 1967
Jenkins, R. A. and Gill, B. E. Determination of Oxides of Nitrogen (NOₓ) in Cigarette smoke by chemiluminescence Analysis. Anal. Chem. 52, (1980) 925-928.
Bodenstein, M, Wachenheim L: Die Geschwindigkeit der Reaktion zwischen Stickoxyd und Sauerstoff. Z. Elektrochem: 24, 183-201, 1918.
UK Department of Health. 1998. Nitric oxide yields of cigarettes. Results for cigarettes sampled in 1996. Commissioned by Smoking policy Unit, 135 Waterloo Road, London SE 1 BUG June 1998
Neurath, G. and Ehmke, H. The nitrate content of Tobacco, Beit Tabakforsch. 2 (7), 333-344 1964.
Broaddus, G., York, J. E. and Moseley, J. M., Factors affecting the levels of nitrate nitrogen in cured tobacco leaves. Tobacco Science 4, 149-157, 1965.
Borland, C, and Higenbottam, T. W. Nitric Oxide Yields of Contemporary UK, US and French Cigarettes. International Journal of Epidemiology, Volume 16, Issue 1, March 1987, Pages 31-34,
Borland, C. D. R., Chamberlain, A. T., Higenbottam, T. W., Barber, R.,W. and Thrush, B. A. A Comparison between the Rate of Reaction of Nitric Oxide in the Gas Phase and in Whole Cigarette Smoke. Beitrii.ge zur Tabakforschung International, Volume 13-No. 2-December 1985
Norman V, Keith C H (1965) Nitrogen oxides in tobacco smoke. Nature 205: 915-916.
V. Norman, A. M. Ihrig, T. M. Larson and B. L. Moss The Effect of Some Nitrogenous Blend Components on NO/NOₓ and HCN Levels in Mainstream and Sidestream Smoke. Beitrage zur Tabakforschung International-Volume 12-No. 2-June 1983
Senneca, O, Solimene, R., Chirone, R. and Salatino, P.:Smoldering Combustion in Cigarette Smoking and Generation of Combustion Byproducts. Environmental Engineering Science 25(10):1389-1398 (2008)
Åkerström S, Mousavi-Jazi M., Klingström J., Leijon, M., Lundkvist, A., and Mirazimi. A., Nitric Oxide Inhibits the Replication Cycle of Severe Acute Respiratory Syndrome Coronavirus.Joumal of Virology, 79.3.1966-1969 (2005).
Darwish, I., Miller C., Kain, K. C., and Conrad Liles, W. Inhaled Nitric Oxide Therapy Fails to Improve Outcome in Experimental Severe Influenza. J Med Sci 2012; 9(2):157-162
Kwonil J., Ashita G., Renukaradhya, Gourapura J; Saif, Linda J. Nitric oxide is elicited and inhibits viral replication in pigs infected with porcine respiratory coronavirus but not porcine reproductive and respiratory syndrome virus. Vet Immunol Immunopathol; 136(3-4): 335-9, 2010.
Houlgate P R, Evans W H., Artifactual chemiluminescence in the determination of nitrogen monoxide in the vapour phase of mainstream cigarette smoke. Analyst. 1989 January; 114(1):71-5.
Klimisch H J, Kirchheim E. Quantitative determination of nitrogen oxide in cigarette smoke by means of chemiluminescence (author's transl) Z Lebensm Unters Forsch. 1977; 163(1):48-52. [Article in German]

The various embodiments described above are provided by way of illustration only and should not be construed to limit the claimed invention. Those skilled in the art will readily recognize various modifications and changes that may be made to the claimed invention without following the example embodiments and applications illustrated and described herein, and without departing from the true spirit and scope of the claimed invention, which is set forth in the following claims.

1. A method of treating a viral pandemic in a patient comprising administering a high dose of nitric oxide (NO) for a single breath to the patient, followed by a period of approximately 5 to 12 breaths of fresh air, where the NO concentration in the single breath is no less than about 1,500 ppm.

2. The method of claim 1, wherein the NO is delivered from a cigarette to which a chemical compound or additive has been added to boost the NO concentration in the inhaled smoke.

3. The method of claim 2, wherein the NO is formed when the cigarette is smoked and the NO is inhaled into the lungs.

4. The method of claim 2, wherein the additive is widely and cheaply available throughout the industrialized world as well as in third world countries.

5. The method of claim 2, wherein the additive or compound includes an inorganic nitrate, for example, including nitrates of potassium, sodium, calcium and ammonia.

6. The method of claim 5, wherein the additive consists of a mixture of nitrate salts.

7. The method of claim 1, wherein all the ingredients are low cost and widely available, including the cigarettes and the additive or additives.

8. The method of claim 5, wherein the nitrate salts are used nitrate fertilizers or in food preparation and preservation.

9. (canceled)

10. The method of claim 2, wherein the additives are sprayed on the cigarette or the cigarette is dipped in a concentrated solution of the additive or a fine powder which is applied to the outside of the cigarette.

11. (canceled)

12. The method of claim 1, wherein the NO concentration that is delivered to the lung is sufficient for NO to enter through the protective wall of a host cell to where the vims is lurking inside the cell, thereby disrupting the replication of the vims inside the cell and slowing down or stopping the infection.

13. A method of treating a subject at risk of infection comprising exposing the subject to an intermittent dose of NO gas, where a relatively massive dose of NO is used to break through the outer protective sheath of host cells where the vims is present, without the high NO concentration causing unacceptably high levels of methemoglobin, since the high NO concentration is for a single breath only, followed by multiple breaths of fresh air to clear some or all of the methemoglobin that may be formed.

14. A method of delivering a life saving drug directly into the lung of a sick patient using a cigarette as a disposable drug delivery engine.

15. A method of claim 14, wherein the dmg is manufactured during the combustion of the additive in the cigarette.

16. A method of claim 15, wherein the dmg is nitric oxide which is manufactured in the cigarette by the decomposition of nitrates in the hot flame zone as the cigarette is smoked.

17. A method of claim 15, wherein the dmg is vaporized and inhaled by the hot smoke as the cigarette is smoked.

18. A treatment device comprising a heat source and a nitric oxide source positioned to be heated by the heat source.

19. A treatment device of claim 18, wherein the heat source is a combustible product.

20. A treatment device of claim 19, wherein the combustible product is a cigarette.

21. A treatment device of claim 18, wherein the nitric oxide source includes an inorganic nitrate.

22. A treatment device of claim 21, wherein the inorganic nitrate is a salt coated on or imbedded in a cigarette.
